(19) United States
(12) Patent Application Publication
(10) Pub. No.: US 2005/0154449 A1
(43) Pub. Date: Jul. 14, 2005

(54) Non-biodegradable drug-eluting sleeves for intravascular devices

(75) Inventor: David R. Elmaleh, Newton, MA (US)

Correspondence Address: GREENBERG TRAURIG, LLP, ONE INTERNATIONAL PLACE, 20th FL, ATTN: PATENT ADMINISTRATOR, BOSTON, MA, 02110, US

(73) Assignee: David Elmaleh

(21) Appl. No.: 10/999,840

(22) Filed: Nov. 29, 2004

Related U.S. Application Data
(63) Continuation-in-part of application No. 10/208,950, filed on Jul. 31, 2002.

(51) Int. Cl. A61F 2/06

(52) U.S. Cl. 623/1.15; 623/1.42; 623/1.44

(57) ABSTRACT

An intravascular device having a removable biocompatible sleeve pulled substantially over the length of the device is provided. The device may be a substantially expandable tubular body defined by a mesh framework. The sleeve can include a plurality of perforations to permit fluid communication with the mesh framework. The sleeve may contain a pharmacotherapeutic agent to permit treatment of certain conditions. After deployment to a site of interest, the device and more specifically, the sleeve, can provide local delivery of sustained or controlled therapeutic dose of the suitable pharmacotherapeutic agent.

### RELATED U.S. APPLICATION(S)

This application is a continuation-in-part of U.S. application Ser. No. 10/208,950, filed Jul. 31, 2002, which application is hereby incorporated herein by reference.

### FIELD OF THE INVENTION

The present invention relates to intravascular devices, and more particularly, to intravascular devices having a non-biodegradable drug-eluting sleeve thereon.

### RELATED ART

Many medical intravascular devices are currently being used either temporarily or permanently inside the human body. One example of an intravascular device includes a stent for use in, for instance, coronary angioplasty. Stents are small mechanical devices, which can be implanted into, for example, a blood vessel to hold open and support the constricted vessel, so as to prevent a re-narrowing or closure of the vessel subsequent to an angioplasty procedure. Stent generally designed to include small metal scaffolds comprising a mesh or perforated tube, for direct insertion to the site of closure or narrowing, and can be mechanically expanded by, for instance, a balloon to reopen the vessel at the site of closure. For proper positioning, stents may be made to permit visualization during and after deployment using imaging techniques such as x-ray radiography and x-ray fluoroscopy. However, due to the nature of the materials used to construct these intravascular devices and their small size, visualization of these devices can often be poor or non-existent.

The mechanical reopening of a constricted vessel with a balloon can sometimes lead to balloon-related injuries of the tissues at the site of closure. Such injuries can often stimulate tissue proliferation at the reopened site during the healing process, and which proliferation can result in pronounced neointimal hyperplasia or restenosis. Restenosis remains the most common post-stenting clinical problem, and requires effective intervention or counter-measures to prevent and/or control its reoccurrence.

Currently, methods for preventing or controlling restenosis are specifically aimed at influencing factors believed to be involved in the body's response to external or internal tissue stimulants, such as angioplasty, stenting procedures, and/or viruses. Common countermeasures which have been used to prevent or control restenosis generally fall into the one of several categories, including (1) mechanical atheroablative techniques, such as debulking, vascular filters, and emboli-trapping devices, (2) ultrasound-initiated atheroablative techniques, (3) light-assisted procedures, predominantly excimer laser angioplasty, (4) pharmacological agents and gene therapy, (5) ultraviolet photophoresis, believed to be an immune modulator, (6) radiation therapy, such as external and endovascular brachytherapy, and (7) re-stenting.

In addition, modifications to stent designs and materials have been proposed to prevent and/or control restenosis. In one approach, non-metallic, biodegradable stent materials, such as high molecular weight Poly-1-lactic acid (PLLA) is used.

Numerous inorganic coatings and surface treatments have also been developed to improve chemical inertness and biocompatibility of metallic stents. Some coatings, such as gold, however, yield a higher rate of in-stent restenosis than uncoated stents. Others, including silicon carbide and turbostatic carbon, show promise but additional studies must be done.

Organic coatings, including both synthetic and natural coatings, have also been widely studied. Among the synthetic coatings studied are Dacron, polyester, polyurethane, polytetrafluoroethylene (PTFE), polyethylacrylate/polymethylmethacrylate, polyvinyl chloride, silicone, collagen, and iridium oxide. Results of studies, such as those with PTFE-coated stents, are disappointing or mixed at best, as there are high occurrences of late thrombo-occlusive events. With only a very few exceptions, the general consensus is that any favorable outcome was not associated with treatment of conventional in-stent restenosis using PTFE-coated stents.

Intracoronary intervention have also been employed to reduce neointima formation by reducing smooth muscle cell proliferation after balloon angioplasty. However, such intervention is often complicated by subacute and late thrombosis. Coronary thrombo-aspiration and coronary pulsed-spray procedures, followed by immediate endovascular therapy, have also been particularly helpful in removing thrombotic material associated with plaque.

In addition, pharmacotherapeutic agents have been used for the treatment of some of the major post-angioplasty complications, including immunosuppresants, anticoagulants and anti-inflammatory compounds, chemotherapy agents, antibiotics, antiallergenic drugs, cell cycle inhibitors, gene therapy compounds, and ceramide therapy compounds. Pharmacotherapeutic agents can be delivered either systemically or locally. Systemic treatment has shown limited success in reducing restenosis following stent implantation, a result believed to be due to inadequate concentration of the pharmacotherapeutic agents at the site of injury. Increased dose administration, however, is constrained by possible systemic toxicity. It has been observed that local delivery of higher doses via drug eluting stents can significantly reduce adverse systemic effects.

Gene therapy have also been employed in the treatment of restenosis. The procedure is directed towards smooth muscle cells and involves gene transfer via DNA, with or without integration of chromosomes, into selected cells. In transduction without integration, the gene is delivered to both cytoplasm and nucleus and is therefore non-selective. Gene transfer for integration employs retrovirus to affect growth stimulators.

Antibiotics, likewise, has been used in the treatment of coronary artery disease. It is known that antibiotics are effective in controlling inflammation caused by a variety of infectious agents found in fatty plaques blocking the arteries. Results of clinical investigation, such as with azithromycin, suggest a modest antibiotic benefits for heart patients.

Similarly, a phospholipid exhibiting immunosuppressive properties, has been shown to block T-cell activation and proliferation, inhibit Taxol-induced cell cycle apoptosis, and activate protein kinase signal translation in malignant myogenic cells. Rapamycin and its analogs exhibit anti-tumor activities at relatively low dose levels, while inducing only mild side effects, an extremely important aspect of patient care.

### SUMMARY OF THE INVENTION

The present invention provides, in one embodiment, an intravascular device, such as a stent, for maintaining an opening within a constricted vessel. The device may also be used for local delivery of at least one pharmacotherapeutic agent to an intravascular site, for the treatment of, for instance, restenosis following, for example, balloon angioplasty.

The intravascular device, in accordance with an embodiment of the invention, includes an expandable substantially tubular body defined by a mesh framework, a removable biocompatible sleeve for pulling over an entire length of the framework. In an embodiment, the sleeve may be flexible and extensible to permit its expansion with the tubular body. The device further includes a plurality of perforations throughout the sleeve to permit communication with the mesh framework. If desired, the sleeve may include a pharmacotherapeutic agent for the treatment or prevention of certain conditions, and may be made from a non-biodegradable polymer.

The device of the present invention may be manufactured by initially providing an expandable substantially tubular body defined by a mesh framework. Thereafter, a biocompatible, flexible and extensible sleeve having opposite open ends, a body portion extending between the open ends, and a plurality of perforations on the body may be expanded at one of the open ends of the sleeve along its diameter. Next one end of the tubular body may be placed within the expanded open end of the sleeve. Once therein, the sleeve may be pulled over the remainder of the tubular body until the sleeve substantially covers the body.

The present invention further provides a kit having, in one embodiment, a removable biocompatible sleeve for positioning over a mesh framework of a substantially tubular intravascular device. The sleeve, in an embodiment, is designed to be flexible and extensible to permit its expansion with the mesh framework, and includes a plurality of perforations to permit communication with the mesh framework.

### BRIEF DESCRIPTION OF THE DRAWINGS

The invention is explained in greater detail below with reference to an exemplary embodiment that is illustrated in the accompanying figures.

FIG. 1 illustrates a sleeved stent for use in accordance with an embodiment of the present invention.

FIG. 2 illustrates a mesh framework defining a stent for use in accordance with an embodiment of the invention.

FIG. 3 illustrates a flexible and extensible sleeve for use with the framework shown in FIG. 2.

FIG. 4 illustrates a stent with a flexible and extensible sleeve around each of the filament of a stent, as set forth in another embodiment of the present invention.

FIG. 5 illustrates a longitudinal view of a sleeve and filament shown in FIG. 4.

FIG. 6 illustrates a sleeved stent in accordance with another embodiment of the present invention.

### DETAILED DESCRIPTION OF SPECIFIC EMBODIMENTS

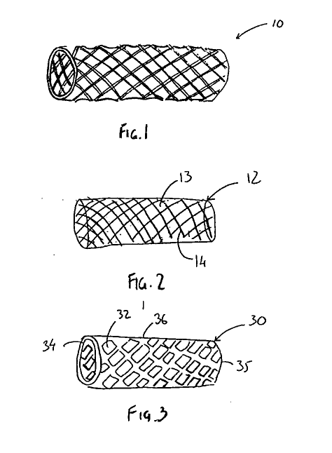

As illustrated in FIG. 1, there is provided, in accordance with an embodiment of the present invention, an intravascular device, such as sleeved stent 10, for maintaining an open lumen in a vascular structure, such as a blood vessel or an artery, and for locally delivering drug to a tissue-injured site caused by, for instance, angioplasty, where over a period of time a therapeutic dose of drug(s) may be released for the treatment of, for example, restenosis.

Previously, local drug delivery to post-angioplasty sites has been accomplished directly from an endovascular catheter. Delivery via an endovascular catheter normally involves delivering a large dose of drug in a very short time period. Because maximum benefits can be achieved by sustained drug delivery, delivery of a large dose in a short time period may not be optimal in many instances.

The stent 10 of the present invention, as shown in FIG. 2, includes, in one embodiment, a substantially tubular mesh framework 12 having openings 13 defined by filaments 14. As the stent 10 will be used to support an opening at a site which was previously closed to maintain a passage therethrough, the mesh framework 12 of stent 10 needs to be made from a material that is sufficiently strong to maintain and support the opening. Although shown in an unexpanded state, the stent 10 can be expanded when positioned at the site of interest. Accordingly, the material from which the framework 12 is made also needs to be sufficiently pliable. In one embodiment of the invention, a material from which the mesh framework 12 may be made includes metal. By providing the stent 10 with a metallic framework 12, the stent 10 may also be visualized, for example, by fluoroscopy during placement of the stent 10 within a vessel.

The stent 10, as shown in FIG. 3 in an expanded state, further includes a sleeve 30 which can be pulled over an entire length of the stent 10. The sleeve 30, in one embodiment, includes opposite open ends 34 and 35, a body portion 36 extending between the open ends, and a plurality of perforations 32 on the body portion 36. The sleeve 30 can be designed so that the body portion 36 extends over the openings 13 and filaments 14 of the entire mesh framework 12 to substantially covered the stent 10. The perforations 32 on the body portion 36 of sleeve 30, in one embodiment, can be designed so that they substantially compliment the openings 13 of stent 10. In this manner, the sleeve 30 may be permeable to, for instance, blood components, and permits communication within the framework 12 if needed. It is believed that presence of perforations 32 can provide proper tissue (e.g., endothial cell) growth at, for example, a post-angioplasty stented site. In addition, the perforations 32 may provide an space through which surrounding tissue may extend therethrough to secure the stent 10 in place. Perforations 32 on the body portion 36 of sleeve 30 may be created by conventional means, including methods involving the use of lasers, mechanical puncturing, or etching.

The sleeve 30, in accordance with an embodiment, may be made from a biocompatible material, so as to minimize toxic reactions from surrounding tissues. Moreover, as the stent 30 will be expanded once placed at the site of constriction, the sleeve 30 can be provided with the ability to be extensible, so as to permit its expansion with the mesh framework 12.

The sleeve 30 may also serve as a storage and direct transport vehicle for the local delivery of, for instance, restenosis-inhibiting pharmaceuticals. For use as a drug-eluting vehicle, the covering sleeve 30 may include a uniform thickness throughout its entire length and may be made from a biodegradable biocompatible polymer. Such a polymer, in one embodiment, may be poly(glycerol-sebacate) or PGS, similar to that described in Wang et al., A tough biodegradable elastomer, Nature 20(6): pp. 602-606 (June 2002), which is hereby incorporated herein by reference. The sleeve 30, according to an embodiment of the present invention, can include at least one of the pharmacotherapeutic agents mentioned above incorporated throughout the sleeve 30 for subsequent local delivery. Alternatively, the pharmacotherapeutic agent or agents can be positioned within each layer of a multiple layer sleeve 30.

Examples of pharmacotherapeutic agents which may be incorporated within the sleeve include Rapamycin, a phospholipid exhibiting immunosuppressive properties. Heparin and glycosaminoglycans are anticoagulants which may be delivered locally after stent implantation. These anticoagulants interact with growth factors and other glycoproteins, which may reduce neointimal proliferation.

Abciximab is a genetically engineered fragment of a chimeric human-murine mono-clonal antibody. It is a glycoprotein inhibitor and works by inhibiting the binding of fibrinogen and other substances to glycoprotein receptor (GBIIb/IIIa) on blood platelets integral to aggregation and clotting. Abciximab appears to be effective in preventing platelet aggregation when used with aspirin and heparin, and appears to be effective in preventing abrupt closure of arteries.

Antibiotics, likewise, can be used in the treatment of coronary artery disease. It is known that antibiotics are effective in controlling inflammation caused by a variety of infectious agents found in fatty plaques blocking the arteries. Azithromycin has been observed to provide modest antibiotic benefits for heart patients.

Other pharmacotherapeutic agents which can be incorporated into the sleeve 30 includes radionuclides for use in the treatment of diseased tissues, and enzymes, which may be encapsulated within a carrier, for instance, a biodegradable sol-gel capsule embedded within the sleeve 30.

It should be appreciated that the concentration of pharmacotherapeutic agent or agents, as well as the rate of degradation of the sleeve 30, can be adjusted according to the treatment for which the stent 10 is being used, so that the rate of release of the agent or agents would be appropriate and sufficient for the treatment.

The sleeved stent 10 of the present invention may be manufactured by initially providing an expandable substantially tubular mesh framework 12. Such a mesh framework 12 can be specially manufactured or commercially obtained from any known source. Thereafter, the biocompatible extensible sleeve 30 may be positioned adjacent the framework 12 and expanded at one of the open ends 34 or 35. Next one end of the tubular framework 12 may be placed within the expanded open end 34 or 35 of the sleeve 30. Once therein, the sleeve 30 may be pulled over the remainder of the periphery of the tubular framework 12 until the sleeve 12 substantially covers the framework 12.

It should be noted that the sleeve 30 may be made to include an outer wall and an inner wall separated by a space (not shown), similar to a glove. In such an embodiment, one end of the framework 12 can be placed within the space between the walls of the sleeve 30 and outer and inner walls of the sleeve 30 can be pulled over both an outer surface of the framework 12 and an inner surface of the framework 12 to completely cover the framework.

In an alternate embodiment, referring now to FIG. 6, there is shown a sleeved stent 60 having a sleeve 61 made from a biocompatible but non-biodegradable material or polymer. Examples of such a material or polymer includes, polyurethane, polyester, polytetrafluoroethylene (PTFE), polyethylacrylate/polymethylmethacrylate, polylactide, polylactide-co-glycolide, polyamides, polydioxanone, polyvinyl chloride, polymeric or silicone rubber, collagen, thermoplastics, a combination thereof, or other materials and/or polymers known in the art. The use of such polymers and/or materials permit the sleeve 61 to be flexible and extensible, so as to allow its placement over the stent 60. In addition, its flexibility and extensibility allows the sleeve 61 to bend or move with the stent 60 as the stent 60 is maneuvered into position within the vessel and subsequently expanded. Furthermore, such sleeve characteristics can enhance the expansion and release of the stent 60 during implantation.

Sleeve 61, similar to sleeve 30, includes opposite open ends 64 and 65, a body portion 66 extending between the open ends, a plurality of perforations 62 on the body portion 66, an exterior surface 67 and an interior surface 68. The sleeve 61 can be designed so that the body portion 66 extends over the openings 63 and filaments of the entire mesh framework to substantially covered the underlying stent 60. The perforations 62 on the body portion 66, in one embodiment, can be designed so that they substantially compliment the openings 63 of stent 60. In this manner, the sleeve 61 may be permeable to, for instance, blood components, and permits communication within the interior of stent 60 if needed. However, it should be appreciated that the perforations 62 may not need to be substantially aligned with the openings 63. Instead, the perforations 62 can be smaller than openings 63 or situated slightly to one side of the openings 63, so that the interior surface 68 of sleeve 61, those areas along the portions between the perforations 62, may be slightly exposed from the perspective of stent 61. Perforations 62 on the body portion 66 of sleeve 61 may be created by conventional means, including methods involving the use of lasers, mechanical puncturing, or etching.

In accordance with an embodiment of the present invention, sleeve 61 can include at least one of the pharmacotherapeutic agents mentioned above. The pharmacotherapeutic agent, such as an anti-restenosis agent, in one embodiment, can be coated onto an outer surface 67 of sleeve 61 using known methodologies, so that the agent can be exposed to the lumen of the vessel within which stent 60 is situated to treat the walls of the vessel from restenosis. Alternatively, sleeve 61 can be coated with an inert biodegradable polymer, such as those provided above, wherein at least one pharmacotherapeutic agent is dispersed therein. As the polymer degrades, the pharmacotherapeutic agent can be released. In an embodiment of the invention, the polymer may be formulate so that degradation may occur from approximately 10 minutes to approximately 30 minutes after expansion of the stent 60.

In another embodiment, the interior surface 68 of the sleeve 61 can be coated directly with a layer of at least one pharmacotherapeutic agent, such as anti-coaglulants, antibodies, or anti-platelet aggregation, or a layer of biodegradable polymer with the pharmacotherapeutic agent dispersed throughout. In this manner, where such surface 68 is exposed from the perspective of the stent 60 and comes into contact with blood flow within the vessel, the presence of pharmacotherapeutic agent can prevent clot formation within the stent.

Sleeve 61, in another embodiment, may include a layer of the pharmacotherapeutic agent within the sleeve 61, or may incorporated the pharmacotherapeutic agent throughout the sleeve 30, along with or independent of the coating on the outer surface 67 and/or interior surface 68 for subsequent local delivery. It should be appreciated that in such an embodiment, the sleeve 61 may include small surface pores (not shown) to facilitate diffusion of the pharmacotherapeutic agent from within the sleeve 61 toward the walls of the vessel and/or toward the interior of the stent where, for instance, blood flow occurs.

By manufacturing a flexible and extensible sleeve, such as sleeve 61, from a biocompatible but non-biodegradable material, for instance, one of the polymers provided above, the failure of stent opening that may be associated with stent having traditional biodegradable polymers can be minimized. In particular, traditional biodegradable polymers that have been coated onto a stent in its collapsed state can bind adjacent filaments of the stent together and/or interfere with pivot points at the intersection between filaments.

In addition, a non-biodegradable sleeve need not be concerned with certain issues faced by a biodegradable sleeve made from traditional polymers. Specifically, sleeves made from traditional polymers, such as biodegradable rubber, may have non-homogenous degradation. Such a non-homogenous degradation pattern can cause breakdown in the polymeric composition, and can lead to earlier-than-desired tear in the polymeric composition at weak points of polymerization. Moreover, the breakdown of the polymeric composition resulting from non-homogenous degradation can generate breakaway pieces from the biodegradable sleeve. These breakaway pieces can move downstream and block fluid flow within the lumen in which the stent is situated.

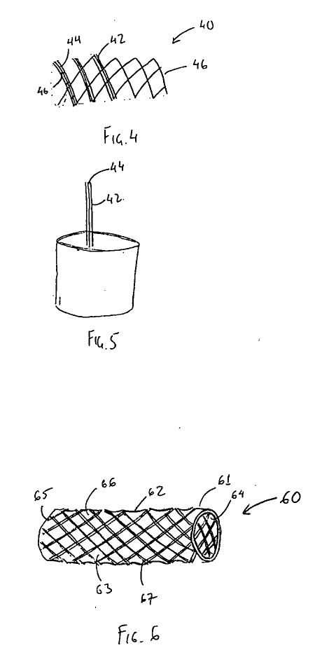

Looking now at FIG. 4, there is illustrated a sleeved stent 40, in accordance with another embodiment of the present invention. Stent 40 is similar to stent 10 described above. However, instead of having a covering sleeve 30 extending over the entire length of the mesh framework 12, stent 40 is providing with a sleeve 42 over each of filaments 44 comprising framework 46. The sleeve 42 may be made from the same polymers as noted above and may include at least one pharmacotherapeutic agent as set forth above.

The covering of each filament 44 by a sleeve 42 may be accomplished using methods known in the art. For example, the framework 46 of stent 40 may be immersed into a polymeric mixture, so as to permit the mixture to be deposited on to each of the filaments 44. The mixture deposited on each filament 44 may subsequently be dried to generate a sleeve 42 thereabout. Alternatively, as shown in FIG. 5, a plurality of individual filaments 44 may be immersed into a polymeric mixture, to deposit the mixture thereon. After the mixture is permitted to dry into a sleeve about each filaments 44, the filaments may be attached to one another to form framework 46, and thus sleeved stent 40. In another embodiment, individual sleeves 42 may also be used to cover each filament 44, which may thereafter by connected to form framework 46.

To enhance the placement of the sleeve 42 about each filament, the sleeve 42 may be attached to each filament 44, for instance, by adhesion. Examples of adhesion protocols which may be used in connection with the present invention includes gluing or by derivatizing the metal filaments along the entire length of the filament 44 or, as shown in FIG. 4, in spaced location.

The present invention further provides a kit having, in one embodiment, a removable biocompatible sleeve, which can either be biodegradable, such as sleeve 30 or non-biodegradable, such as sleeve 60, for positioning over a mesh framework of a substantially tubular intravascular device. The sleeve 30 or 60, in an embodiment, is designed to be extensible to permit its expansion with the mesh framework, and includes a plurality of perforations to permit communication with the mesh framework. The sleeve 30 or 60 can be made to include at least one pharmacotherapeutic agent. In use, one end of sleeve 30 or 60 may be expanded and placed over the mesh framework of the device. Once thereon, the sleeve 30 or 60 may be pulled over the remainder of the periphery of the framework until the sleeve 30 or 60 substantially covers the framework.

Alternative, a kit can be provided to include a polymeric mixture for forming a biodegradable, biocompatible polymeric solution. The kit can also include a mechanism for applying the solution on to a framework and/or filaments of a tubular intravascular device, so as to cover each of the filaments.

The sleeved stent of the present invention may be used to maintain an opening within a variety of different vessels. For instance, the sleeved stent may be placed within a coronary artery or a carotid artery. The sleeved stent may also be used to constrict a passageway, for instance, the coronary sinus, among others. To constrict a passageway, the sleeve on the stent may be made so that it is substantially resistant to expansion, so as to permit the sleeve to exert a force on the tubular framework to constrict the tubular framework. The sleeved stent may also be used as a renal stent, gastrointestinal stent, radiation and chemotherapy stent.

While the invention has been described in connection with the specific embodiments thereof, it will be understood that it is capable of further modification. For instance, the sleeve may be used to cover non-metallic stents and may be made with multiple layers, each with a different rate of degradation. The sleeve 30 may also be adapted for use with other intravascular devices for implantation within a patient's body. Furthermore, this application is intended to cover any variations, uses, or adaptations of the invention, including such departures from the present disclosure as come within known or customary practice in the art to which the invention pertains, and as fall within the scope of the appended claims.

1. An intravascular device comprising:
an expandable substantially tubular body defined by a mesh framework;
a removable non-biodegradable biocompatible sleeve for positioning over the mesh framework, the sleeve being flexible and extensible to permit expansion and movement with the body;
a layer of a pharmacotherapeutic agent on the sleeve; and
a plurality of perforations positioned on the sleeve to permit communication with the mesh framework.

2. A device as set forth in claim 1, wherein the mesh framework includes openings between filaments defining the framework, which openings substantially compliment the perforations on the sleeve.

3. A device as set forth in claim 1, wherein the biocompatible sleeve includes a substantially uniform thickness along the entire sleeve.

4. A device as set forth in claim 1, wherein the biocompatible sleeve is made from a polymer.

5. A device as set forth in claim 4, wherein the polymer includes at least one of polyurethane, polyester, polytetrafluoroethylene (PTFE), polyethylacrylate/polymethylmethacrylate, polylactide, polylactide-co-glycolide, polyamides, polydioxanone, polyvinyl chloride, polymeric or silicone rubber, collagen, thermoplastics, or a combination thereof.

6. A device as set forth in claim 1, wherein the biocompatible sleeve is constructed so that it minimizes failure of the expansion of the mesh framework.

7. A device as set forth in claim 1, wherein the pharmacotherapeutic agent includes at least one of an immunosuppressant, an antibiotic, a cell cycle inhibitor, an anti-inflammatory, an anticoagulant, an antiplatelet aggregation, an antibody, an antiallergen, and a gene therapy and a ceramide therapy compound.

8. A device as set forth in claim 1, wherein the layer of pharmacotherapeutic agent includes a coating of at least on pharmacotherapeutic agent on an outer surface of the sleeve.

9. A device as set forth in claim 8, further including a coating of at least one pharmacotherapeutic agent on an interior surface of the sleeve.

10. A device as set forth in claim 8, further including a coating of a biodegradable polymer having at least one pharmacotherapeutic agent dispersed therein on an interior surface of the sleeve.

11. A device as set forth in claim 1, wherein the layer of pharmacotherapeutic agent includes a coating of a biodegradable polymer having the pharmacotherapeutic agent dispersed therein on an outer surface of the sleeve.

12. A device as set forth in claim 11, further including a coating of at least one pharmacotherapeutic agent on an interior surface of the sleeve.

13. A device as set forth in claim 11, further including a coating of a biodegradable polymer having at least one pharmacotherapeutic agent dispersed therein on an interior surface of the sleeve.

14. A device as set forth in claim 1, wherein the layer of pharmacotherapeutic agent includes a layer within the sleeve.

15. A devices as set forth in claim 1, wherein the layer of pharmacotherapeutic agent includes a mixture of the agent throughout the sleeve.

16. A method of preparing an intravascular device, the method comprising:
providing an expandable substantially tubular body defined by a mesh framework;
providing a non-biodegradable, biocompatible, flexible and extensible sleeve having opposite open ends, a body portion extending between the open ends, a layer of a pharmacotherapeutic agent on the body, and a plurality of perforations on the body;
expanding at least one open end of the sleeve along its diameter;
placing one end of the tubular body within the expanded open end of the sleeve; and
pulling the sleeve over the remainder of the tubular body until the sleeve substantially covers the tubular body.

17. A method as set forth in claim 11, further including positioning the plurality of perforations on the body of the sleeve in such a way that when the mesh framework is in an expanded state, the perforations on the sleeve are substantially aligned with openings in the mesh framework.

18. A kit comprising:
a removable non-biodegradable biocompatible sleeve for positioning over a mesh framework of a substantially tubular intravascular device,
the sleeve being flexible and extensible to permit expansion and movement with the mesh framework, and including
a layer of a pharmacotherapeutic agent; and
a plurality of perforation to permit communication with the mesh framework.
